(19) United States
(12) Plant Patent Application Publication
(10) Pub. No.: US 2003/0084491 P1
(43) Pub. Date: May 1, 2003

(54) Spray rose plant named 'Meiptipier'

(76) Inventor: Alain A. Meilland, Antibes (FR)

Correspondence Address: BURNS DOANE SWECKER & MATHIS L L P, POST OFFICE BOX 1404, ALEXANDRIA, VA, 22313-1404, US

(21) Appl. No.: 09/984,848

(22) Filed: Oct. 31, 2001

(51) Int. Cl. A01H 5/00

(52) U.S. Cl. PLT/143

(57) ABSTRACT

A new and distinct variety of rose plant of the Spray Class is provided which abundantly forms attractive double bicolored blossoms that are deep rosy pink on the inner petals and pale ivory pink on the outer petals. The buds are globose in configuration and are borne on stems having relatively few thorns. An open vase-shaped growth habit is exhibited. The foliage is matt medium to dark green and contrasts nicely with the bicolored pink blossoms. The new variety is well suited for growing as a distinctive gift plant in containers.

### SUMMARY OF THE INVENTION

The new variety of Rosa hybrida spray rose plant was created by artificial pollination wherein two parents were crossed which previously had been studied in the hope that they would contribute the desired characteristics. The female parent (i.e., the seed parent) of the new variety was the product of the cross of the ‘Internikip’ variety (U.S. Plant Pat. No. 8,114) and ‘Olijdum’ variety (U.S. Plant patent application Ser. No. 09/293,176, filed Apr. 16, 1999). The male parent (i.e., the pollen parent) of the new variety was the product of the cross of the ‘Meichevil’ variety (U.S. Plant Pat. No. 7,334) and the ‘Keinoumi’ variety (U.S. Plant Pat. No. 8,484). The parentage of the new variety can be summarized as follows:

(‘Interniki’×‘Olijdum’) ×(‘Meichevil’×‘Keinoumi’).

The seeds resulting from the above pollination were sown and small plants were obtained which were physically and biologically different from each other. Selective study resulted in the identification of a single plant of the new variety.

It was found that the new variety of spray rose plant of the present invention possesses the following combination of characteristics:

(a) forms generally globose buds in the form of a spray on stems having relatively few thorns,

(b) forms in abundance attractive double bicolored blossoms that are deep rosy pink on the central petals and pale ivory pink on the outer petals,

(c) is a vigorous grower and exhibits an open vase-shaped growth habit, and

(d) forms attractive matt medium to dark green foliage upon maturity.

The bicolored spray inflorescence coloration contrasts nicely with the dense medium to dark green foliage.

The new variety well meets the needs of the horticultural industry. It is suited for use in the production of cut floral sprays under greenhouse growing conditions or for growing as ornamentation in the garden. The new variety is particularly well suited for growing as a distinctive gift plant in containers.

The new variety has been found to undergo asexual propagation in France and at West Grove, Pa. by the rooting of cuttings. Asexual propagation by the use of cuttings as performed in France and at West Grove, Pa. has shown that the characteristics of the new variety are strictly transmissible from one generation to another.

The new variety has been named the ‘Meiptipier’ variety.

### BRIEF DESCRIPTION OF THE PHOTOGRAPHS

The accompanying photographs show as nearly true as it is reasonably possible to make the same, in color illustrations of this character, typical specimens of the new variety. The rose plants of the new variety were approximately eighteen months of age and were observed during October while growing in containers on their own roots in greenhouses at West Grove, Pa.

FIG. 1—illustrates a spray of buds in various stages of opening as well as the mature foliage that displays a matt finish. Anthocyanin coloration is apparent on some of the younger leaflets that are included in the photograph as well as on the margins of maturing leaflets.

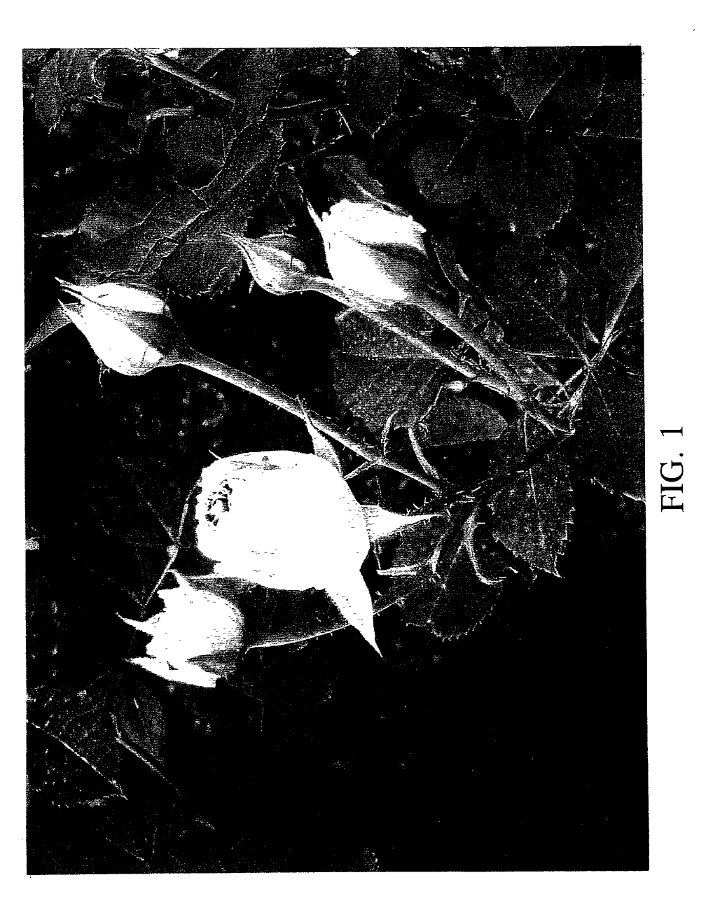

FIG. 2—illustrates at the right a close view of an open flower. The bicoloration of the petals is shown wherein the central petals are a deep rosy pink and the outer petals are a pale ivory pink. Typical buds and foliage are included in the photograph.

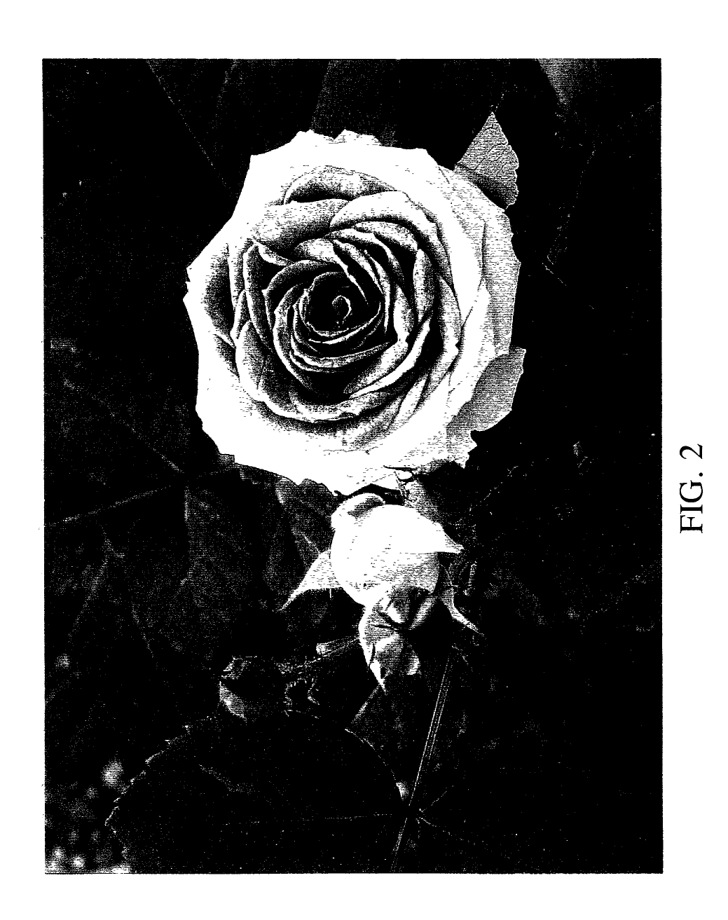

### DETAILED DESCRIPTION

The chart used in the identification of the colors is that of The Royal Horticultural Society (R.H.S. Colour Chart). When more common color terms are utilized they are to be accorded customary dictionary significance. The description is based on eighteen month-old specimens of the new variety which were observed during October while growing in containers on their own roots in greenhouses at West Grove, Pa.

Class: Spray.

Plant:

height.—commonly assumes a height of approximately 57 to 83 cm at the end of the growing season.

habit.—open and vase-shaped.

Branches:

thorns.—size: average approximately 0.7 cm in length. quantity: few (approximately 1.6 prickles per linear inch from the base of current growth to the first flowering bud) on flowering stems and frequently present on the ventral side of the rachis. configuration: somewhat straight upper surface and slightly concave under surface. color: prickles on the terminal three inches of a young shoot bear a noticeable reddish-pink coloration of Red-Purple Group 63A to 63B.

Leaves:

leaflets.—number: 3, 5, and 7 (most often) in a ratio of approximately 1:2:3. shape: generally elliptic with a generally symmetrical acute tip and an obtuse base. texture: typical and normal. serration: simple and regular (as illustrated). general appearance: medium to dark green with a matt finish when fully mature. The newest immature leaflets display a glossy surface that later becomes semi-glossy with some maturity. color (young foliage): upper surface: with some reddish anthocyanin coloration particularly at the margins. color (adult foliage): upper surface: near Green Group 139A. under surface: near Greyed-Green Group 191B to 191C with margins commonly approaching Greyed-Green Group 191A.

Inflorescene

number of flowers.—approximately 1 to 8 per stem and commonly 4 to 5 per stem.

sepals.—shape: with elongated tips (as illustrated) and commonly bear some thin extensions (as illustrated).

buds.—shape: globose.

flower.—form: cup-shaped when fully open. diameter: approximately 4.5 to 6 cm and approximately 5 cm on average. color: the central petals are primarily Red-Purple Group 66C with tones of Red-Purple Group 67C and 68A. As the blossoms reach full maturity, the central petal coloration lightens somewhat to near Red-Purple Group 62A towards the margins and to near White Group 155A and 155B with some light pink tones near the base. The outer petals are pale ivory pink, White Group 155A and 155B with shades of light pink. The lightest colored petals are present towards the outside of the blooms causing the fully open blooms to display a bicolored appearance when viewed from above. fragrance: slight. petal number: approximately 27 to 40 with an average of approximately 32. petal configuration: with lightly reflexed edges. petaloids: approximately 5 to 11 small petaloids with an average of approximately 7 are typically present per bloom. Such petaloids sometimes appear to be fused to the anthers. petal drop: good, the petals commonly detach cleanly. lasting quality: long, approximately seven to ten days.

Development

vegetation.—strong.

blooming.—abundant.

resistance to diseases.—good under standard greenhouse growing conditions.

aptitude to bear fruit.—none observed with plants having been closely pruned to date.

I claim:

1. A new and distinct variety of spray rose plant characterized by the following combination of characteristics:
(a) forms generally globose buds in the form of a spray on stems having relatively few thorns,
(b) forms in abundance attractive double bicolored blossoms that are deep rosy pink on the central petals and pale ivory pink on the outer petals,
(c) exhibits an open vase-shaped growth habit, and
(d) forms attractive matt medium to dark green foliage upon maturity; substantially as herein shown and described.
